SEQUENCE LISTING (11 sequences)

SEQ ID NO: 1 - protein, 40 aa, Artificial Sequence
  Synthetic
XXEGTFTSDXSXXXEXXAXXXFIXWLXXXXXXXXXXXXXX

SEQ ID NO: 2 - protein, 32 aa, Artificial Sequence
  Protein
XXEGTFTSDVSXYLEXXAAXEFIXWLVXXXXX

SEQ ID NO: 3 - protein, 39 aa, Artificial Sequence
  protein
HGEGTFTSDLSKQMEEEAVRLFIEWLKNGGPSSGAPPPS

SEQ ID NO: 4 - protein, 44 aa, Artificial Sequence
  protein
HGEGTFTSDLSKQMEEEAVRLFIEWLKNGGPSSGAPPSKKKKKK

SEQ ID NO: 5 - protein, 31 aa, Artificial Sequence
  protein
HXEGTFTSDVSSYLEXQAAKEFIAWLVKXRK

SEQ ID NO: 6 - protein, 31 aa, Artificial Sequence
  protein
HXEGTFTSDVSSYLEGQAAKEFIAWLVRGRG

SEQ ID NO: 7 - protein, 30 aa, Artificial Sequence
  protein
HXEGTFTSDVSSYLEGQAAKEFIAWLVKXR

SEQ ID NO: 8 - protein, 60 aa, Artificial Sequence
  protein
HGEGTFTSDVSSYLEGQAAKEFIAWLVKGRHGEGTFTSDVSSYLEGQAAKEFIAWLVKGR

SEQ ID NO: 9 - protein, 31 aa, Artificial Sequence
  protein
HGEGTFTSDLSKQMEEEAVRLFIEWLKNGGX

SEQ ID NO: 10 - protein, 40 aa, Artificial Sequence
  Synthetic
HXXGTFITSDLSKQMEEEAVRLFIEWLKNGGPSSGAPPPS

SEQ ID NO: 11 - protein, 40 aa, Artificial Sequence; identical to SEQ ID NO: 10
  Synthetic

(19) United States
(12) Patent Application Publication
(10) Pub. No.: US 2015/0038417 A1
(43) Pub. Date: Feb. 5, 2015

(54) GLP-1 Agonists

(71) Applicant: Novo Nordisk A/S, Bagsvaerd (DK)

(72) Inventor: Kirsten Raun, Lyngby (DK)

(73) Assignee: Novo Nordisk A/S

(21) Appl. No.: 14/363,117

(22) PCT Filed: Dec. 10, 2012
(86) PCT No.: PCT/EP2012/074896

§ 371 Date: Jun. 5, 2014

Related U.S. Application Data
(60) Provisional application No. 61/578,572, filed on Dec. 21, 2011.

(30) Foreign Application Priority Data
Dec. 9, 2011 (EP) 11192912.1
Jul. 6, 2012 (EP) 12175404.8

(51) Int. Cl. A61K 38/26; A61K 45/06

(52) U.S. Cl. CPC A61K 38/26; A61K 45/06
USPC 514/11.7

(57) ABSTRACT

A method for the prevention or treatment of alcoholism and drug addiction comprising administering to a subject in need thereof a therapeutically effective amount of a GLP-1 agonist.

The present invention relates to the use of GLP-1 agonists for the treatment or prevention of alcoholism and drug addiction.

### BACKGROUND

Compulsive and uncontrolled consumption of alcoholic beverages can lead to alcoholism, where the person has a physical dependence on alcohol and continues to drink alcohol despite problems with physical health, mental health, and social, family, or job responsibilities. Excess alcohol intake (alcohol abuse) can also lead to similar problems even without alcohol dependence.

Both alcoholism and alcohol disease are medically considered to be diseases. The World Health Organisation estimates that there are 140 million people with alcoholism worldwide. The biological mechanisms that cause alcoholism are not well understood, but research suggests that certain genes may increase the risk of alcoholism. However, the genes which could be linked with alcoholism are not known.

Alcoholism is called a “dual disease” since it includes both mental and physical components, such as social environment, stress, mental health, family, history, age, ethnic group, and gender, all influence the risk for the condition. Alcohol damages almost every organ in the body, including the brain. Long-term alcohol abuse produces changes in the brain's chemical structure, with results such as tolerance and physical dependence. These changes maintain the person with alcoholism's compulsive inability to stop drinking and result in alcohol withdrawal syndrome if the person stops. The cumulative toxic effects of chronic alcohol abuse can cause both medical and psychiatric problems.

The drug Antabuse® (disulfiram) is used to support the treatment of chronic alcoholism by producing an acute sensitivity to alcohol. After alcohol intake under the influence of disulfiram, the concentration of acetaldehyde in the blood increases to a level which is higher than that present when alcohol alone is metabolised. Acetaldehyde is one of the major causes of the symptoms of a “hangover” which results in a negative reaction to alcohol intake and the patient experiencing the effects of a severe hangover including symptoms of, for example, accelerated heart rate, shortness of breath, nausea and vomiting.

Naltrexone is an opioid receptor antagonist used primarily in the management of alcohol dependence and opioid dependence. It is marketed in generic form as its hydrochloride salt, naltrexone hydrochloride. Naltrexone is a competitive antagonist for opioid receptors, effectively blocking the effects of endorphins and opiates. Naltrexone is used to decrease cravings for alcohol and encourage abstinence. Alcohol causes the body to release endorphins, which in turn release dopamine and activate the reward pathways; hence when naltrexone is in the body there is a reduction in the pleasurable effects from consumption. While some patients do well with the oral formulation, there is a drawback in that it must be taken daily, and a patient whose craving becomes overwhelming can obtain opiate euphoria simply by skipping a dose before resuming abuse.

Nalmefene can be taken with alcohol and is being tested as a way to reduce a person's craving for drink. The drug works by blocking a craving mechanism regulated by the brain's opioid receptors.

In addition to alcohol, drugs are known to cause addiction. Addiction is a primary, chronic, neurobiological disease, with genetic, psychosocial, and environmental factors influencing its development and manifestations. It is characterised by behaviours that include one or more of the following: impaired control over drug use, compulsive use, continued use despite harm, and craving.

Drugs which are known to cause addiction include both legal and illegal drugs as well as prescription and over-the-counter drugs. For example, the following drugs are known to cause addiction: stimulants such as amphetamine, methamphetamine, cocaine and caffeine; sedatives and hypnotics such as alcohol, barbiturates and benzodiazepines; opiate and opioid analgesics such as morphine and codeine; opiates, such as heroin and fully synthetic opioids, such as methadone.

Psychological counseling and rehabilitation centres are time consuming and expensive and there is a great risk of relapse. There is a need for methods to prevent or treat dependence and addiction of drugs and alcohol.

### SUMMARY

In one embodiment the invention relates to a method for the prevention or treatment of alcoholism or drug addiction comprising administering to a subject in need thereof a therapeutically effective amount of a GLP-1 agonist. In one embodiment the invention relates to a GLP-1 agonist for use in the prevention or treatment of alcoholism or drug addiction. In one embodiment the invention relates to a pharmaceutical composition comprising a GLP-1 agonist for use in the prevention or treatment of alcoholism or drug addiction.

### DESCRIPTION OF THE INVENTION

According to the present invention, there is provided a method for the prevention or treatment of alcoholism comprising administering to a subject in need thereof a therapeutically effective amount of a GLP-1 agonist.

According to a further embodiment of the present invention, there is also provided a method for the prevention or treatment of drug addiction comprising administering to a subject in need thereof a therapeutically effective amount of a GLP-1 agonist.

According to a further embodiment of the present invention, there is also provided a method for the prevention or treatment of alcoholism comprising administering to a subject in need thereof a therapeutically effective amount of a GLP-1 agonist and simultaneously or sequentially administering another agent.

In one embodiment, wherein the therapeutic agent is for the treatment of alcoholism and is selected from the group consisting of: disulfiram, calcium carbimide, naltrexone, nalmefene, acamprosate, and benzodiazepines such as diazepam.

According to a further embodiment of the present invention, there is also provided a method for the prevention or treatment of drug addiction comprising administering to a subject in need thereof a therapeutically effective amount of a GLP-1 agonist and simultaneously or sequentially administering another therapeutic agent.

In one embodiment, wherein the therapeutic agent is for the treatment of drug addiction and is selected from the group consisting of: stimulants such as amphetamine, methamphetamine, cocaine and caffeine; sedatives and hypnotics such as alcohol, barbiturates and benzodiazepines; opiate and opioid analgesics such as morphine and codeine; opiates such as heroin and fully synthetic opioids such as methodone.

In one embodiment, the GLP-1 agonist is a GLP-1 peptide.

Conveniently, the GLP-1 peptide comprises the amino acid sequence of the formula (I):

Formula (I)

Xaa₇-Xaa₈-Glu-Gly-Thr-Phe-Thr-Ser-Asp-Xaa₁₆-Ser-
Xaa₁₈-Xaa₁₉Xaa₂₀GluXaa₂₂-Xaa₂₃-Ala-Xaa₂₅-Xaa₂₆-
Xaa₂₇-Phe-Ile-Xaa₃o-Trp-Leu-Xaa₃₃-Xaa₃₄-Xaa₃₅-
Xaa₃₆-Xaa₃₇-Xaa₃₈-Xaa₃₉-Xaa₄o-Xaa₄₁-Xaa₄₂-Xaa₄₃-
Xaa₄₄-Xaa₄₅-Xaa46

wherein

Xaa₇ is L-histidine, D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;

Xaa₈ is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;

Xaa₁₆ is Val or Leu;

Xaa₁₈ is Ser, Lys or Arg;

Xaa₁₉ is Tyr or Gin;

Xaa₂₀ is Leu or Met;

Xaa₂₂ is Gly, Glu or Aib;

Xaa₂₃ is Gin, Glu, Lys or Arg;

Xaa₂₅ is Ala or Val;

Xaa₂₆ is Lys, Glu or Arg;

Xaa₂₇ is Glu or Leu;

Xaa₃₀ is Ala, Glu or Arg;

Xaa₃₃ is Val or Lys;

Xaa₃₄ is Lys, Glu, Asn or Arg;

Xaa₃₅ is Gly or Aib;

Xaa₃₆ is Arg, Gly or Lys;

Xaa₃₇ is Gly, Ala, Glu, Pro, Lys, amide or is absent;

Xaa₃₈ is Lys, Ser, amide or is absent;

Xaa₃₉ is Ser, Lys, amide or is absent;

Xaa₄₀ is Gly, amide or is absent;

Xaa₄₁ is Ala, amide or is absent;

Xaa₄₂ is Pro, amide or is absent;

Xaa₄₃ is Pro, amide or is absent;

Xaa₄₄ is Pro, amide or is absent;

Xaa₄₅ is Ser, amide or is absent;

Xaa₄₆ is amide or is absent;

provided that if Xaa₃₈, Xaa₃₉, Xaa₄₀, Xaa₄₁, Xaa₄₂, Xaa₄₃, Xaa₄₄, Xaa₄₅ or Xaa₄₆ is absent then each amino acid residue downstream is also absent.

In one embodiment, the GLP-1 peptide comprises the amino acid sequence of formula (II):

Formula (II)

Xaa₇-Xaa₈-Glu-Gly-Thr-Phe-Thr-Ser-Asp-Val-Ser-
Xaa₁₈-Tyr-Leu-Glu-Xaa22-Xaa₂₃-Ala-Ala-Xaa₂₆-Glu-
Phe-Ile-Xaa₃ₒ-Trp-Leu-Val-Xaa₃₄-Xaa₃₅-Xaa₃₆-
Xaa₃₇Xaa₃₈

wherein

Xaa₇ is L-histidine, D-histidine, desamino-histidine, 2-amino-histidine,-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;

Xaa₁₈ is Ser, Lys or Arg;

Xaa₂₂ is Gly, Glu or Aib;

Xaa₂₃ is Gln, Glu, Lys or Arg;

Xaa₂₆ is Lys, Glu or Arg; Xaa30 is Ala, Glu or Arg;

Xaa₃₄ is Lys, Glu or Arg;

Xaa₃₅ is Gly or Aib;

Xaa₃₆ is Arg or Lys;

Xaa₃₇ is Gly, Ala, Glu or Lys;

Xaa₃₈ is Lys, amide or is absent.

Conveniently, the GLP-1 peptide is selected from GLP-1 (7-35), GLP-1 (7-36), GLP-1 (7-36)-amide, GLP-1 (7-37), GLP-1 (7-38), GLP-1 (7-39), GLP-1 (7-40), GLP-1 (7-41) or an analogue thereof.

In one embodiment, the GLP-1 peptide comprises no more than fifteen amino acid residues which have been exchanged, added or deleted as compared to GLP-1 (7-37), or no more than ten amino acid residues which have been exchanged, added or deleted as compared to GLP-1 (7-37).

Optionally, the GLP-1 peptide comprises no more than six amino acid residues which have been exchanged, added or deleted as compared to GLP-1 (7-37).

Conveniently, the GLP-1 peptide comprises no more than 4 amino acid residues which are not encoded by the genetic code.

In one embodiment, the GLP-1 peptide is a DPPIV protected GLP-1 peptide.

Optionally, the GLP-1 peptide is DPPIV stabilised.

Conveniently, the GLP-1 peptide comprises an Aib residue in position 8.

In one embodiment, the amino acid residue in position 7 of said GLP-1 peptide is selected from the group consisting of D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine and 4-pyridylalanine.

Optionally, the GLP-1 peptide is selected from the group consisting of Arg³⁴GLP-1 (7-37), Lys³⁸Arg^{26,34}GLP-1 (7-38), Lys³⁸Arg^{26,34}GLP-1 (7-38)-OH, Lys³⁶Arg^{26,34}GLP-1 (7-36),

Aib^{8,22,35}GLP-1 (7-37), Aib^{8,35}GLP-1 (7-37), Aib^{8,22}GLP-1 (7-37),

Aib^{8,22,35}Arg^{26,34}Lys³⁸GLP-1(7-38), Aib^{8,35}Arg^{26,34}Lys³⁸GLP-1 (7-38),

Aib^{8,22}Arg^{26,34}Lys³⁸GLP-1 (7-38), Aib^{8,22,35}Arg^{26,34}Lys³⁸GLP-1 (7-38),

Aib^{8,35}Arg^{26,34}Lys³⁸GLP-1 (7-38), Aib^{8,22,35}Arg²⁶Lys³⁸GLP-1(7-38),

Aib^{8,35}Arg²⁶Lys³⁸GLP-1 (7-38), Aib^{8,22}Arg²⁶Lys³⁸GLP-1 (7-38),

Aib^{8,22,35}Arg³⁴Lys³⁸GLP-1 (7-38), Aib^{8,35}Arg³⁴Lys³⁸GLP-1 (7-38),

Aib^{8,22}Arg³⁴Lys³⁸GLP-1 (7-38), Aib^{8,22,35}Ala³⁷Lys³⁸GLP-1(7-38),

Aib^{8,35}Ala³⁷Lys³⁸GLP-1(7-38), Aib^{8,22}Ala³⁷Lys³⁸GLP-1 (7-38),

Aib^{8,22,35}Lys³⁷GLP-1 (7-37), Aib^{8,35}Lys³⁷GLP-1 (7-37) and Aib^{8,22}Lys³⁷GLP-1 (7-38).

Conveniently, the GLP-1 peptide is attached to said hydrophilic spacer via the amino acid residue in position 23, 26, 34, 36 or 38 relative to the amino acid sequence of GLP-1 (7-37).

In one embodiment, the GLP-1 peptide is exendin-4, an exendin-4-analogue, or a derivative of exendin-4.

Optionally, the GLP-1 peptide comprises the amino acid sequence of the following formula:

H-His-Gly-Glu-Gly-Thr-Phe-Thr-
Ser-Asp-Leu-Ser-Lys-Gln-Met-
Glu-Glu-Glu-Ala-Val-Arg-Leu
Phe-Ile-Glu-Trp-Leu-Lys-Asn-
Gly-Gly-Pro-Ser-Ser-Gly-Ala-
Pro-Pro-Pro-Ser-NH₂

In one embodiment, the GLP-1 peptide is ZP-10, i.e.

HGEGTFTSDLSKQMEEEAVRLFIEWLKNGGPSSGAPPSKKKKKK-amide.

Conveniently, wherein one albumin binding residue via said hydrophilic spacer is attached to the C-terminal amino acid residue of said GLP-1 peptide.

In one embodiment, wherein a second albumin binding residue is attached to an amino acid residue which is not the C-terminal amino acid residue.

In one embodiment, wherein the GLP-1 peptide is selected from the group consisting of liraglutide, semaglutide, taspoglutide, albiglutide and dulaglitide.

In one embodiment, wherein the GLP-1 peptide is TTP054.

In one embodiment, wherein the GLP-1 peptide has the following structure:

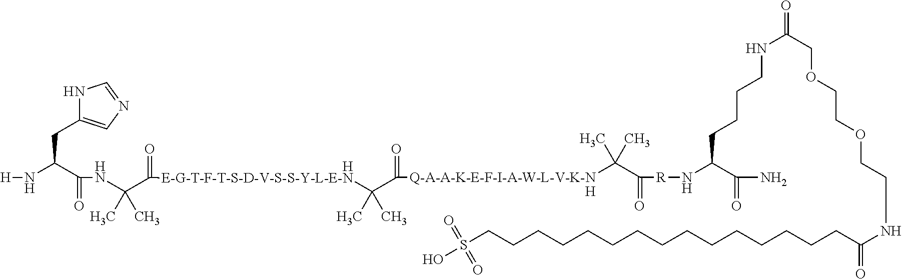

In one embodiment, wherein the GLP-1 peptide has the following structure:

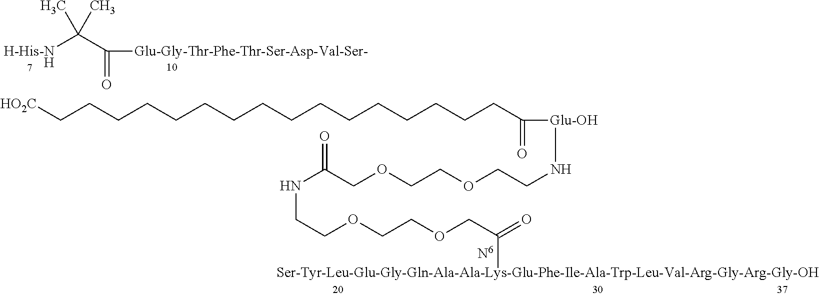

In one embodiment, wherein the GLP-1 peptide has the following structure:

His-Aib-Glu-Gly-Thr-Phe-Thr-Ser-Asp-Val-Ser-Ser-
Tyr-Leu-Glu-Gly- Gln-Ala-Ala-Lys-Glu-Phe-Ile-Ala-
Trp-Leu-Val-Lys-Aib-Arg

In one embodiment, wherein the GLP-1 peptide has the following structure:

(His-Gly-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg )2-

genetically fused to human albumin.

In one embodiment, wherein the GLP-1 peptide is dulaglitide.

Optionally, wherein the addiction comprises addiction to a drug selected from the group consisting of: stimulants such as amphetamine, methamphetamine, cocaine and caffeine; sedatives and hypnotics such as alcohol, barbiturates and benzodiazepines; opiate and opioid analgesics such as morphine and codeine; opiates such as heroin and fully synthetic opioids such as methadone.

According to a further embodiment of the present invention there is provided a GLP-1 agonist is for use in the prevention or treatment of alcoholism.

According to a further embodiment of the present invention there is provided a GLP-1 agonist for use in the prevention or treatment of drug addiction.

According to a further embodiment of the present invention there is provided a pharmaceutical composition comprising a GLP-1 agonist for use in the prevention or treatment of alcoholism.

According to a further embodiment of the present invention there is provided a pharmaceutical composition comprising a GLP-1 agonist for use in the prevention or treatment of drug addiction.

According to a further embodiment of the present invention there is provided a pharmaceutical composition for use comprising administering to a subject in need thereof a therapeutically effective amount of a GLP-1 agonist and simultaneously or sequentially administering another agent.

According to a further embodiment of the present invention there is provided a pharmaceutical composition for use wherein the therapeutic agent is for the treatment of alcoholism and is selected from the group consisting of: disulfiram, calcium carbimide, naltrexone, nalmefene, acamprosate, and benzodiazepines such as diazepam.

According to a further embodiment of the present invention there is provided a pharmaceutical composition for use comprising administering to a subject in need thereof a therapeutically effective amount of a GLP-1 agonist and simultaneously or sequentially administering another therapeutic agent.

According to a further embodiment of the present invention there is provided a pharmaceutical composition for use, wherein the therapeutic agent is for the treatment of drug addiction and is selected from the group consisting of: stimulants such as amphetamine, methamphetamine, cocaine and caffeine; sedatives and hypnotics such as alcohol, barbiturates and benzodiazepines; opiate and opioid analgesics such as morphine and codeine; opiates such as heroin and fully synthetic opioids such as methodone.

It has been found by the present applicant that treatment with GLP-1 agonists e.g. liraglutide will influence the compulsive and uncontrolled consumption of alcoholic beverages in a manner that will lead to more controlled alcohol intake i.e. a reduction in alcohol consumption for which the individual's intake is no longer compulsive and uncontrolled. This results in improved physical and mental health problems, associated with excess alcohol consumption. Treatment with GLP-1 agonists can also be used to treat drug addiction.

Treatment with a GLP-1 agonist e.g. a once daily injection with liraglutide is convenient and safe and will reduce the compulsive and uncontrolled urge for both alcohol and drug intake, for the benefit of patients with alcoholism or drug addiction as well as for families, friends and employees.

In the present context, “alcoholism” implies in broad terms problems with alcohol and is generally used to mean compulsive and uncontrolled consumption of alcoholic beverages. For the purposes of the present invention, the term “alcoholism” can be split into two further terms “alcohol abuse” and “alcohol dependency”. Alcohol abuse is the repeated use of alcohol despite recurrent adverse consequences. Alcohol dependence is alcohol abuse combined with tolerance, withdrawal, and an uncontrollable drive to drink.

In the present context, “drug addiction” implies when an individual persists in the use of one or more drugs despite problems related to use of the substance. Compulsive and repetitive use may result in tolerance to the effect of the drug and withdrawal symptoms when use is reduced or stopped.

An “effective amount” of a compound as used herein means an amount sufficient to cure, alleviate, or partially arrest the clinical manifestations of a given disease or state and its complications. An amount adequate to accomplish this is defined as “effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary. In one embodiment, “effective amount” may be referred to as “therapeutically effective amount”.

The term “treatment” and “treating” as used herein means the management and care of a patient for the purpose of combating a condition, such as a disease or a disorder.

The term is intended to include the full spectrum of treatments for a given condition from which the patient is suffering, such as administration of the active compound to alleviate the symptoms or complications; to delay the progression of the disease, disorder, or condition; to alleviate or relieve the symptoms and complications; and/or, to cure or eliminate the disease, disorder, or condition as well as to prevent the condition. Prevention is to be understood as the management and care of a patient for the purpose of combating the disease, condition, or disorder and includes the administration of the active compounds to prevent the onset of the symptoms or complications.

In the present context, “subject” is intended to indicate a human that is currently suffering from alcoholism, alcohol dependency or drug addiction.

In the present context, “drug regimen” is intended to mean the administration of a drug within its prescribed parameters of timing (e.g., once daily, twice daily, once weekly, etc.) and amount.

The term “hydrophilic spacer” as used herein means a spacer that separates a peptide and an albumin binding residue with a chemical moiety which comprises at least 5 non-hydrogen atoms where 30-50% of these are either N or O.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids.

Natural amino acids which are not encoded by the genetic code are e.g. hydroxyproline, γ-carboxyglutamate, ornithine, phosphoserine, D-alanine and D-glutamine.

Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), p-alanine, 3-aminomethyl benzoic acid, anthranilic acid.

The term “analogue” as used herein referring to a polypeptide means a modified pep-tide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A simple system is used to describe analogues: For example Arg³⁴GLP-1 (7-37) Lys designates a GLP-1 analogue wherein the naturally occurring lysine at position 34 has been substituted with arginine and a lysine residue has been added to the C-terminal (position 38). Formulae of peptide analogues and derivatives thereof are drawn using standard single letter abbreviation for amino acids used according to IUPAC-IUB nomenclature.

The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. An example of a derivative of GLP-1(7-37) is N^{ε26}-(γ-Glu(N^{α}-hexadecanoyl))-[Arg³⁴, Lys²⁵]) GLP-1 (7-37).

The term “GLP-1 peptide” as used herein means GLP-1 (7-37), a GLP-1 analogue, a GLP-1 derivative or a derivative of a GLP-1 analogue.

The term “exendin-4 peptide” as used herein means exendin-4 (1-39), an exendin-4 analogue, an exendin-4 derivative or a derivative of an exendin-4 analogue.

The term “DPP-IV protected” as used herein referring to a polypeptide means a polypeptide which has been chemically modified in order to render said compound resistant to the plasma peptidase dipeptidyl aminopeptidase-4 (DPP-IV). The DPP-IV enzyme in plasma is known to be involved in the degradation of several peptide hormones, e.g. GLP-1, Exendin-4 etc. Thus a considerable effort is being made to develop analogues and derivative of the polypeptides susceptible to DPP-IV mediated hydrolysis in order to reduce the rate of degradation by DPP-IV.

The term “simultaneous” as used herein means in the same therapeutic intervention i.g. two tablets given together, or both drugs in one IV bag.

The term “sequential” used herein means in the same therapeutic window (e.g. in a 24, 12, 6, 4 or 2 hour period etc).

In the present context, “a GLP-1 agonist” is understood to refer to any compound, including peptides and non-peptide compounds, which fully or partially activate the human GLP-1 receptor. In one embodiment, the “GLP-1 agonist” is any peptide or non-peptide small molecule that binds to a GLP-1 receptor, such as with an affinity constant (K_{D}) or a potency (EC₅₀) of below 1 μM, e.g. below 100 nM as measured by methods known in the art (see e.g., WO 98/08871).

Methods for identifying GLP-1 agonists are described in WO 93/19175 (Novo Nordisk A/S) and examples of suitable GLP-1 analogues and derivatives which can be used according to the present invention includes those referred to in WO 2005/027978 (Novo Nordisk A/S), the teachings of which are both incorporated by reference herein.

In yet another embodiment the GLP-1 agonist is a stable GLP-1 analogue/-derivative. Throughout this application a “stable GLP-1 analogue/derivative” means a GLP-1 analogue or a derivative of a GLP-1 analogue which exhibits an in vivo plasma elimination half-life of at least 10 hours in man, as determined by the method described below. Examples of stable GLP-1 analogue/derivatives can be found in WO 98/08871, WO 99/43706, WO 02/46227 and WO 2005/027978. In one embodiment the stable GLP-1 analogue/-derivative exhibits an in vivo plasma elimination half-life in manof at least 10 hours, such as at least 20 hours or at least 60 hours, e.g. determined by the method described below. In one embodiment astable GLP-1 analogue/-derivative may be referred to as a long acting GLP-1 agonist.

The method for determination of plasma elimination half-life of a compound in man is as follows: The compound is dissolved in an isotonic buffer, pH 7.4, PBS or any other suitable buffer. The dose is injected peripherally, such as in the abdominal or upper thigh. Blood samples for determination of active compound are taken at frequent intervals, and for a sufficient duration to cover the terminal elimination part (e.g., Pre-dose, 1, 2, 3, 4, 5, 6, 7, 8, 10, 12, 24 (day 2), 36 (day 2), 48 (day 3), 60 (day 3), 72 (day 4) and 84 (day 4) hours post dose). Determination of the concentration of active compound is performed as described in Wilken et al., Diabetologia 43 (51), 2000. Derived pharmacokinetic parameters are calculated from the concentration-time data for each individual subject by use of non-compartmental methods, using the commercially available software WinNonlin Version 2.1 (Pharsight, Cary, N.C., USA). The terminal elimination rate constant is estimated by log-linear regression on the terminal log-linear part of the concentration-time curve, and used for calculating the elimination half-life.

The GLP-1 agonist may be formulated so as to have a half-life in man, as discussed above, of at least 10 hours. This may be obtained by sustained release formulations known in the art.

In one embodiment the GLP-1 agonist is administered in an amount and at a frequency which provides chronic plasma exposure. As used herein the term “chronic plasma exposure” when used in connection with a GLP-1 agonist is intended to mean continuous plasma exposure of a therapeutically effective amount of said GLP-1 agonist. In one embodiment an example of chronic plasma exposure is the plasma exposure obtained after once daily administration of a GLP-1 agonist having an in vivo plasma elimination half-life in man of at least 10 hours.

In yet another embodiment, the GLP-1 agonist is exendin-4 or exendin-3, an exendin-4 or exendin-3 analogue, or a derivative of any of these. Examples of exendins as well as analogues, derivatives, and fragments thereof to be included within the present invention are those disclosed in WO 97/46584, U.S. Pat. No. 5,424,286, and WO 01/04156, the teachings of which are incorporated herein by reference. The exendin polypeptides disclosed include HGEGTFTSDLSKQMEEEAVRLFIEWLKNGGX; wherein X=P or Y, and HX1X2GTFITS DLSKQMEEEAVRLFIEW LKNGGPSSGAPPPS; wherein XIX2=SD (exendin-3) or GE (exendin-4). WO 97/46584 describes truncated versions of exendin peptides.

In an embodiment of the invention, the GLP-1 agonist does not include GLP-1, exendin-3 or exendin-4.

The present invention also encompasses pharmaceutically acceptable salts of the GLP-1 agonists. Such salts include pharmaceutically acceptable acid addition salts, pharmaceutically acceptable metal salts, ammonium, and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates which the present GLP-1 agonists are able to form.

Peptide GLP-1 compounds can be produced by appropriate derivatization of an appropriate peptide backbone which has been produced by recombinant DNA technology or by peptide synthesis (e.g., Merrifield-type solid phase synthesis) as known in the art of peptide synthesis and peptide chemistry.

The route of administration of GLP-1 agonists may be any route which effectively transports the active compound to the appropriate or desired site of action, such as oral, nasal, buccal, pulmonal, transdermal, or parenteral.

Medicaments or pharmaceutical compositions containing a GLP-1 agonist such as liraglutide may be administered parenterally to a patient in need thereof. Parenteral administration may be performed by subcutaneous, intramuscular or intravenous injection by means of a syringe, optionally a pen-like syringe.

Alternatively, parenteral administration can be performed by means of an infusion pump. A further option is a composition which may be a powder or a liquid for the administration of a GLP-1 agonist in the form of a nasal or pulmonal spray. As a still further option, the GLP-1 agonist can also be administered transdermally, e.g., from a patch, optionally an iontophoretic patch, or transmucosally, e.g., bucally. The above-mentioned possible ways to administer GLP-1 agonists are not considered as limiting the scope of the invention.

In one embodiment, a GLP-1 agonist is co-administered together with a further therapeutically active compound used in the treatment of alcoholism and drug addiction.

### Pharmaceutical Compositions

One object of the present invention is to provide a pharmaceutical formulation comprising a compound according to the present invention which is present in a concentration from about 0.1 mg/ml to about 25 mg/ml, and wherein said formulation has a pH from 2.0 to 10.0. The pharmaceutical formulation may comprise a compound according to the present invention which is present in a concentration from about 0.1 mg/ml to about 50 mg/ml, and wherein said formulation has a pH from 2.0 to 10.0. The formulation may further comprise a buffer system, preservative(s), isotonicity agent(s), cheating agent (s), stabilizers and surfactants.

In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further embodiment the invention relates to a pharmaceutical formulation comprising an aqueous solution of a compound according to the present invention, and a buffer, wherein said compound is present in a concentration from 0.1 mg/ml or above, and wherein said formulation has a pH from about 2.0 to about 10.0.

In a further embodiment the invention relates to a pharmaceutical formulation comprising an aqueous solution of a compound according to the present invention, and a buffer, wherein said compound is present in a concentration from 0.1 mg/ml or above, and wherein said formulation has a pH from about 7.0 to about 8.5.

In a further embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0. In one embodiment, the pH of the formulation is at least 1 pH unit from the isoelectric point of the compound according to the present invention, such as the pH of the formulation is at least 2 pH units from the isoelectric point of the compound according to the present invention.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, hepes, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, ethanol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 30 mg/ml. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG 400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one OH group and includes, for example, mannitol, sorbitol, inositol, galacititol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention.

The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof.

In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the cheating agent is present in a concentration from 2 mg/ml to 5 mg/ml. Each one of these specific cheating agents constitutes an alternative embodiment of the invention. The use of a cheating agent in pharmaceutical compositions is well-known to the skilled person. For convenience reference is made to Remington: The Science and Practice of Pharmacy, 19^{th} edition, 1995.

In a further embodiment of the invention the formulation further comprises a stabiliser. The use of a stabilizer in pharmaceutical compositions is well-known to the skilled per-son. For convenience reference is made to Remington: The Science and Practice of Pharmacy, 19th edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38: 48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18: 1169-1206; and Mumenthaler et al (1994) Pharm. Res. 11:12-20), or air drying (Carpenter and Crowe (1988) Cryobiology 25: 459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” it is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids used for preparing the compositions of the invention are those carrying a charged side chain, such as arginine, lysine, aspartic acid, and glutamic acid. In one embodiment, the amino acid used for preparing the compositions of the invention is glycine.

Any stereoisomer (i.e. L or D) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cystein analogues include S-methyl-L cystein.

As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulfuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D or a mixture thereof) can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabiliser selected from the group of high molecular weight polymers or low molecular compounds. In a further embodiment of the invention the stabilizer is selected from polyethylene glycol (e.g. PEG 3350), polyvinylalcohol (PVA), polyvinylpyrrolidone, carboxyhydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutical active polypeptide therein. Stabilizing agents of particular interest to the present invention include, but are not limited to, methionine and EDTA, which protect the polypeptide against methionine oxidation, and a non-ionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic F68, poloxamer 188 and 407, Triton X-100), polyoxyethylene sorbitan fatty acid esters, starshaped PEO, polyoxyethylene and polyethylene derivatives such as alkylated and alkoxylated derivatives (tweens, e.g. Tween-20, Tween-40, Tween-80 and Brij-35), polyoxyethylene hydroxystearate, monoglycerides or ethoxylated derivatives thereof, diglycerides or polyoxyethylene derivatives thereof, alcohols, glycerol, lecitins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether) derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives-(e.g. sodium tauro-dihydrofusidate etc.), long-chain fatty acids and salts thereof C6-C12 (eg. oleic acid and caprylic acid), acylcarnitines and derivatives, N′X-acylated derivatives of lysine, arginine or histidine, or side-chain acylated derivatives of lysine or arginine, N-acylated derivatives of dipeptides comprising any combination of lysine, arginine or histidine and a neutral or acidic amino acid, N-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N, N-dimethyl-3-ammonio-1-propanesulfonate, anionic (alkyl-aryl-sulphonates) monovalent surfactants, zwitterionic surfactants (e.g. N-alkyl-N, N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

A composition for parenteral administration of GLP-1 compounds may, for example, be prepared as described in WO 03/002136.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine).

Such additional ingredients, of course, should not adversely affect the overall stability of the pharmaceutical formulation of the present invention.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in-situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly (vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions thereof, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semi-solids, powder and solutions for pulmonary administration of the compound, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. In one embodiment controlled release and sustained release systems are administered subcutaneous. Without limiting the scope of the invention, examples of useful controlled release system and compositions are hydrogels, oleaginous gels, liquid crystals, polymeric micelles, microspheres, nanoparticles. In one embodiment the composition comprises injectable polymer-based microspheres.

Methods to produce controlled release systems useful for compositions of the current invention include, but are not limited to, crystallization, condensation, co-cystallization, precipitation, co-precipitation, emulsification, dispersion, high pressure homogenization, encapsulation, spray drying, microencapsulation, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, in-traperitoneal or intravenous injection by means of a syringe, optionally a pen-like syringe.

Alternatively, parenteral administration can be performed by means of an infusion pump. A further option is a composition which may be a solution or suspension for the administration of the compound according to the present invention in the form of a nasal or pulmonal spray.

As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces.

Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person.

Physical stability of the aqueous protein formulations can also be evaluated by using a spectroscopic agent or probe of the conformational status of the protein. The probe may be a small molecule that may bind to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolyse to form a free carboxylic acid.

Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahem. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

Pharmaceutical compositions containing a GLP-1 agonist according to the present invention may be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the GLP-1 derivative in the form of a nasal or pulmonal spray. As a still further option, the GLP-1 derivatives of the invention can also be administered transdermally, e.g. from a patch, optionally a iontophoretic patch, or transmucosally, e.g. bucally.

Thus, the injectable compositions of the GLP-1 agonist of the invention can be prepared using the conventional techniques of the pharmaceutical industry which involves dissolving and mixing the ingredients as appropriate to give the desired end product.

According to one procedure, the GLP-1 agonist is dissolved in an amount of water which is somewhat less than the final volume of the composition to be prepared. An isotonic agent, a preservative and a buffer is added as required and the pH value of the solution is adjusted if necessary using an acid, e.g. hydrochloric acid, or a base, e.g. aqueous sodium hydroxide as needed. Finally, the volume of the solution is adjusted with water to give the desired concentration of the ingredients.

Further to the above-mentioned components, solutions containing a GLP-1 agonist according to the present invention may also contain a surfactant in order to improve the solubility and/or the stability of the GLP-1 agonist.

A composition for nasal administration of certain peptides may, for example, be prepared as described in European Patent No. 272097 (to Novo Nordisk A/S) or in WO 93/18785.

The particular GLP-1 agonist to be used and the optimal dose level for any patient will depend on the disease to be treated and on a variety of factors including the efficacy of the specific peptide derivative employed, the age, body weight, physical activity, and diet of the patient, on a possible combination with other drugs, and on the severity of the case. It is recommended that the dosage of the GLP-1 agonist of this invention be determined for each individual patient by those skilled in the art.

In another embodiment the present invention relates to a compound according to the present invention for use in the prevention or treatment of alcoholism and drug addiction.

The present invention relates to the use of a compound according to the invention for the preparation of a medicament for the prevention or treatment of alcoholism and drug addiction.

### EMBODIMENTS OF THE INVENTION

The following are non-limiting embodiments of the invention:

1. A method for the prevention or treatment of alcoholism comprising administering to a subject in need thereof a therapeutically effective amount of a GLP-1 agonist.
2. A method for the prevention or treatment of drug addiction comprising administering to a subject in need thereof a therapeutically effective amount of a GLP-1 agonist.
3. A method for the prevention or treatment of alcoholism comprising administering to a subject in need thereof a therapeutically effective amount of a GLP-1 agonist and simultaneously or sequentially administering another therapeutic agent.
4. The method according to Embodiment 3 wherein the therapeutic agent is for the treatment of alcoholism and is selected from the group consisting of: disulfiram, calcium carbimide, naltrexone, nalmefene, acamprosate, and benzodiazepines such as diazepam.
5. A method for the prevention or treatment of drug addiction comprising administering to a subject in need thereof a therapeutically effective amount of a GLP-1 agonist and simultaneously or sequentially administering another therapeutic agent.
6. The method according to Embodiment 5 wherein the therapeutic agent is for the treatment of drug addiction and is selected from the group consisting of: stimulants such as amphetamine, methamphetamine, cocaine and caffeine; sedatives and hypnotics such as alcohol, barbiturates and benzodiazepines; opiate and opioid analgesics such as morphine and codeine; opiates such as heroin and fully synthetic opioids such as methodone.
7. The method according to any one of Embodiments 1 to 6, wherein the GLP-1 agonist is a GLP-1 peptide.
8. The method according to Embodiment 7, wherein the GLP-1 peptide comprises the amino acid sequence of the formula (I):

Formula (I)

Xaa₇Xaa₈-Glu-Gly-Thr-Phe-Thr-Ser-Asp-Xaa₁₆-Ser-
Xaa₁₈-Xaa₁₉Xaa₂₀GluXaa₂₂-Xaa₂₃-Ala-Xaa₂₅-Xaa₂₆-
Xaa₂₇-Phe-Ile-Xaa₃o-Trp-Leu-Xaa₃₃-Xaa₃₄-Xaa₃₅-
Xaa₃₆-Xaa₃₇-Xaa₃₈-Xaa₃₉-Xaa₄o-Xaa₄₁-Xaa₄₂-Xaa₄₃-
Xaa₄₄-Xaa₄₅-Xaa₄₆

wherein

Xaa₇ is L-histidine, D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;

Xaa₈ is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;

Xaa₁₆ is Val or Leu;

Xaa₁₈ is Ser, Lys or Arg;

Xaa₁₉ is Tyr or Gln;

Xaa₂₀ is Leu or Met;

Xaa₂₂ is Gly, Glu or Aib;

Xaa₂₃ is Gin, Glu, Lys or Arg;

Xaa₂₅ is Ala or Val;

Xaa₂₆ is Lys, Glu or Arg;

Xaa₂₇ is Glu or Leu;

Xaa₃₀ is Ala, Glu or Arg;

Xaa₃₃ is Val or Lys;

Xaa₃₄ is Lys, Glu, Asn or Arg;

Xaa₃₅ is Gly or Aib;

Xaa₃₆ is Arg, Gly or Lys;

Xaa₃₇ is Gly, Ala, Glu, Pro, Lys, amide or is absent;

Xaa₃₈ is Lys, Ser, amide or is absent;

Xaa₃₉ is Ser, Lys, amide or is absent;

Xaa₄₀ is Gly, amide or is absent;

Xaa₄₁ is Ala, amide or is absent;

Xaa₄₂ is Pro, amide or is absent;

Xaa₄₃ is Pro, amide or is absent;

Xaa₄₄ is Pro, amide or is absent;

Xaa₄₅ is Ser, amide or is absent;

Xaa₄₆ is amide or is absent;

provided that if Xaa₃₈, Xaa₃₉, Xaa₄₀, Xaa₄₁, Xaa₄₂, Xaa₄₃, Xaa₄₄, Xaa₄₅ or Xaa₄₆ is absent then each amino acid residue downstream is also absent.

9. The method according to Embodiment 7, wherein said polypeptide is a GLP-1 peptide comprising the amino acid sequence of formula (II):

Formula (II)

Xaa₇-Xaa₈-Glu-Gly-Thr-Phe-Thr-Ser-Asp-Val-Ser-
Xaa₁₈-Tyr-Leu-Glu-Xaa22-Xaa₂₃-Ala-Ala-Xaa₂₆-Glu-
Phe-Ile-Xaa₃ₒ-Trp-Leu-Val-Xaa₃₄-Xaa₃₅-Xaa₃₆-
Xaa₃₇Xaa₃₈

wherein

Xaa₇ is L-histidine, D-histidine, desamino-histidine, 2-amino-histidine,-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;

Xaa₁₈ is Ser, Lys or Arg;

Xaa₂₂ is Gly, Glu or Aib;

Xaa₂₃ is Gln, Glu, Lys or Arg;

Xaa₂₆ is Lys, Glu or Arg; Xaa30 is Ala, Glu or Arg;

Xaa₃₄ is Lys, Glu or Arg;

Xaa₃₅ is Gly or Aib;

Xaa₃₆ is Arg or Lys;

Xaa₃₇ is Gly, Ala, Glu or Lys;

Xaa₃₈ is Lys, amide or is absent.

10. The method according to Embodiment 7 wherein said GLP-1 peptide is selected from GLP-1 (7-35), GLP-1 (7-36), GLP-1 (7-36)-amide, GLP-1 (7-37), GLP-1 (7-38), GLP-1 (7-39), GLP-1 (7-40), GLP-1 (7-41) or an analogue thereof.
11. The method according to Embodiment 7 wherein said GLP-1 peptide comprises no more than fifteen amino acid residues which have been exchanged, added or deleted as compared to GLP-1 (7-37), or no more than ten amino acid residues which have been exchanged, added or deleted as compared to GLP-1 (7-37).
12. The method according to Embodiment 7, wherein said GLP-1 peptide comprises no more than six amino acid residues which have been exchanged, added or deleted as compared to GLP-1 (7-37).
13. The method according to Embodiment 7, wherein said GLP-1 peptide comprises no more than 4 amino acid residues which are not encoded by the genetic code.
14. The method according to Embodiment 7, wherein said GLP-1 peptide is a DPPIV protected GLP-1 peptide.
15. The method according to Embodiment 7, wherein GLP-1 peptide is DPPIV stabilised.
16. The method according to Embodiment 7, wherein said GLP-1 peptide comprises an Aib residue in position 8.
17. The method according to any one Embodiments 7 to 16, wherein the amino acid residue in position 7 of said GLP-1 peptide is selected from the group consisting of D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine and 4-pyridylalanine.
18. The method according to any one of Embodiments 7 to 16, wherein said GLP-1 peptide is selected from the group consisting of Arg³⁴GLP-1 (7-37), Lys³⁸Arg^{26,34}GLP-1 (7-38), Lys³⁸Arg^{26,34}GLP-1 (7-38)-OH, Lys³⁶Arg^{26,34}GLP-1 (7-36),

Aib^{8,22,35}GLP-1 (7-37), Aib^{8,35}GLP-1 (7-37), Aib^{8,22}GLP-1 (7-37),

Aib^{8,22,35}Arg^{26,34}Lys³⁸GLP-1(7-38), Aib^{8,35}Arg^{26,34}Lys³⁸GLP-1 (7-38),

Aib^{8,22}Arg^{26,34}Lys³⁸GLP-1 (7-38), Aib^{8,22,35}Arg^{26,34}Lys³⁸GLP-1 (7-38),

Aib^{8,35}Arg^{26,34}Lys³⁸GLP-1 (7-38), Aib^{8,22,35}Arg²⁶Lys³⁸GLP-1(7-38),

Aib^{8,35}Arg²⁶Lys³⁸GLP-1 (7-38), Aib^{8,22}Arg²⁶Lys³⁸GLP-1 (7-38),

Aib^{8,22,35}Arg³⁴Lys³⁸GLP-1 (7-38), Aib^{8,35}Arg³⁴Lys³⁸GLP-1 (7-38),

Aib^{8,22}Arg³⁴Lys³⁸GLP-1 (7-38), Aib^{8,22,35}Ala³⁷Lys³⁸GLP-1(7-38),

Aib^{8,35}Ala³⁷Lys³⁸GLP-1(7-38), Aib^{8,22}Ala³⁷Lys³⁸GLP-1 (7-38),

Aib^{8,22,35}Lys³⁷GLP-1 (7-37), Aib^{8,35}Lys³⁷GLP-1 (7-37) and Aib^{8,22}Lys³⁷GLP-1 (7-38).

19. The method according to any one of Embodiments 7 to 16, wherein said GLP-1 peptide is attached to said hydrophilic spacer via the amino acid residue in position 23, 26, 34, 36 or 38 relative to the amino acid sequence of GLP-1 (7-37).
20. The method according to Embodiment 7, wherein the GLP-1 peptide is exendin-4, an exendin-4-analogue, or a derivative of exendin-4.
21. The method according to Embodiment 20 wherein the GLP-1 peptide comprises the amino acid sequence of the following formula:

H-His-Gly-Glu-Gly-Thr-Phe-Thr-
Ser-Asp-Leu-Ser-Lys-Gln-Met-
Glu-Glu-Glu-Ala-Val-Arg-Leu
Phe-Ile-Glu-Trp-Leu-Lys-Asn-
Gly-Gly-Pro-Ser-Ser-Gly-Ala-
Pro-Pro-Pro-Ser-NH₂

22. The method according to Embodiment 7, wherein said GLP-1 peptide is ZP-10, i.e. HGEGTFTSDLSKQMEEEAVRLFIEWLKNGGPSSGAPPSKKKKKK-amide.
23. The method according to Embodiment 7 wherein one albumin binding residue via said hydrophilic spacer is attached to the C-terminal amino acid residue of said GLP-1 peptide.
24. The method according to Embodiment 23, wherein a second albumin binding residue is attached to an amino acid residue which is not the C-terminal amino acid residue.
25. The method according to Embodiment 7, wherein the GLP-1 peptide is selected from the group consisting of liraglutide, semaglutide, taspoglutide, albiglutide and dulaglitide.
26. The method according to Embodiment 7, wherein the GLP-1 peptide is TTP054.
27. The method according to Embodiment 7, wherein the GLP-1 peptide has the following structure:

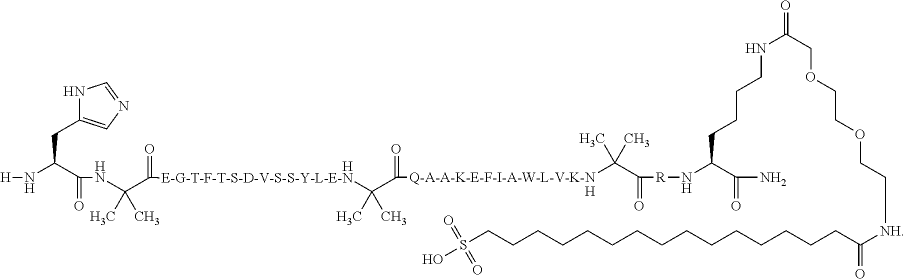

28. The method according to Embodiment 7, wherein the GLP-1 peptide has the following structure:

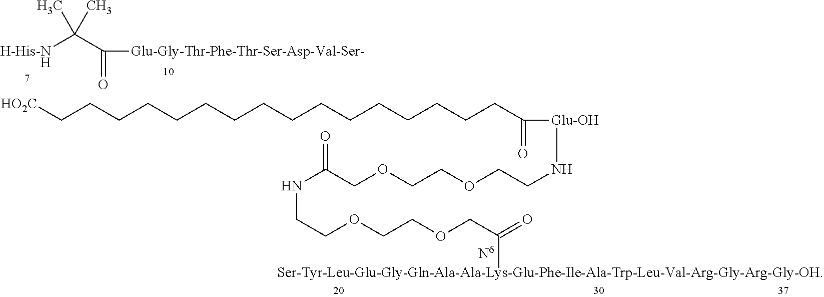

29. The method according to Embodiment 7, wherein the GLP-1 peptide has the following structure: His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg.
30. The method according to Embodiment 7, wherein the GLP-1 peptide has the following structure: (His-Gly-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg)2-genetically fused to human albumin.
31. The method according to Embodiment 7 wherein the GLP-1 peptide is dulaglitide.
32. The method according to Embodiment 2, wherein the drug addiction comprises addiction to a drug selected from the group consisting of: stimulants such as amphetamine, methamphetamine, cocaine and caffeine; sedatives and hypnotics such as alcohol, barbiturates and benzodiazepines; opiate and opioid analgesics such as morphine and codeine; opiates such as heroin and fully synthetic opioids such as methadone.
33. A GLP-1 agonist for use in the prevention or treatment of alcoholism.
34. A GLP-agonist for use according to Embodiment 33, wherein the GLP-1 agonist is as defined in any of Embodiments 7 to 32.
35. A GLP-1 agonist for use in the prevention or treatment of drug addiction.
36. A GLP-1 agonist for use according to Embodiment 35, wherein the GLP-1 agonist is as defined in any of Embodiments 7 to 32.
37. A pharmaceutical composition comprising a GLP-1 agonist for use in the prevention or treatment of alcoholism.
38. A pharmaceutical composition for use according to Embodiment 37, wherein the GLP-1 agonist is as defined in any of Embodiments 7 to 32.
39. A pharmaceutical composition comprising a GLP-1 agonist for use in the prevention or treatment of drug addiction.
40. A pharmaceutical composition for use according to Embodiment 39, wherein the GLP-1 agonist is as defined in any of Embodiments 7 to 32.
41. A pharmaceutical composition for use according to Embodiment 37, wherein the use comprises administering to a subject in need thereof a therapeutically effective amount of a GLP-1 agonist and simultaneously or sequentially administering another agent.
42. A pharmaceutical composition for use according to Embodiment 41 wherein the therapeutic agent is for the treatment of alcoholism and is selected from the group consisting of: disulfiram, calcium carbimide, naltrexone, nalmefene, acamprosate, and benzodiazepines such as diazepam.
43. A pharmaceutical composition for use according to Embodiment 39, wherein the use comprises administering to a subject in need thereof a therapeutically effective amount of a GLP-1 agonist and simultaneously or sequentially administering another therapeutic agent.
44. A pharmaceutical composition for use according to Embodiment 43, wherein the therapeutic agent is for the treatment of drug addiction and is selected from the group consisting of: stimulants such as amphetamine, methamphetamine, cocaine and caffeine; sedatives and hypnotics such as alcohol, barbiturates and benzodiazepines; opiate and opioid analgesics such as morphine and codeine; opiates such as heroin and fully synthetic opioids such as methodone.

### EXAMPLES

### Example 1

### Preparation of GLP-1 Compounds

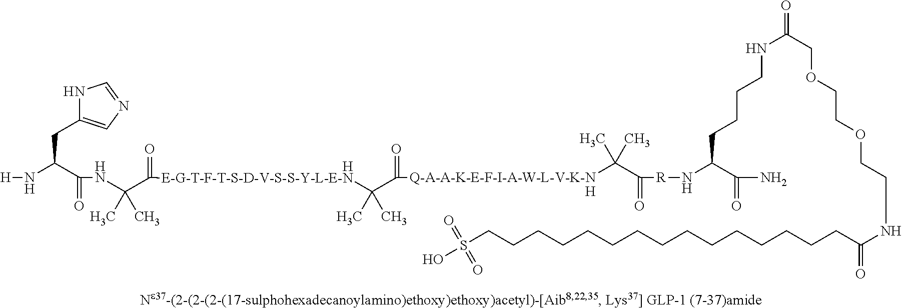

The above compound was prepared in accordance with the following method. A resin (Rink amide, 0.68 mmol/g Novabiochem 0.25 mmole) was used to produce the primary sequence on an AB1433A machine according to manufacturer's guidelines. All protecting groups were acid labile with the exception of the residue used in position 37 (FmocLys (ivDde)-OH, Novabiochem) allowing specific deprotection of this lysine rather than any other lysine.

The above prepared resin (0.25 mmole) containing the GLP-1 analogue amino acid sequence was placed in a manual shaker/filtration apparatus and treated with 2% hydrazine in N-methyl pyrrolidone in (2×12 min. 2×20 ml) to remove the Dde group. The resin was washed with N-methyl pyrrolidone (4×20 ml). Fmoc-8-amino-3,6-dioxaoctanoic acid (Neosystem FA03202) (4 molar equivalents relative to resin) was dissolved in N-methyl pyrroldone/methylene chloride (1:1, 20 ml). Hydroxybenzotriazole (HOBt) (4 molar equivalents relative to resin) and diisopropylcarbodiimide (4 molar equivalents relative to resin) was added and the solution was stirred for 15 min. The solution was added to the resin and diisopropylethylamine (4 molar equivalents relative to resin) was added. The resin was shaken 24 hours at room temperature. The resin was washed with N-methyl pyrrolidone (4×20 ml). A solution of 20% piperidine in N-methyl pyrrolidone (3×20 ml, 10 min each) was added to the resin while shaking. The resin was washed with N-methyl pyrrolidone (4×20 ml).

Dodecanoic acid (4 molar equivalents relative to resin) was dissolved in N-methyl pyrroldone/methylene chloride (1:1, 20 ml). Hydroxybenzotriazole hydrate (HOBt;H₂0) (4 molar equivalents relative to resin) and diisopropylcarbodiimide (4 molar equivalents relative to resin) were added and the solution was stirred for 15 min. The solution was added to the resin and diisopropylethylamine (4 molar equivalents relative to resin) was added. The resin was shaken 24 hours at room temperature. The resin was washed with N-methyl pyrrolidone (2×20 ml), N-methyl pyrrolidone/methylene chloride (1:1) (2×20 ml) and methylene chloride (2×20 ml). The peptide was cleaved from the resin by stirring for 180 min at room temperature with a mixture of trifluoroacetic acid, water and triisopropylsilane (95:2.5:2.5 15 ml). The cleavage mixture was filtered and the filtrate was concentrated to an oil in vacuum. The crude peptide was precipitated from this oil with 45 ml diethyl ether and washed 3 times with 45 ml diethyl ether. The crude peptide was purified by preparative HPLC on a 20 mm×250 mm column packed with 7μ C-18 silica. The crude peptide was dissolved in 5 ml 50% acetic acid in water and diluted to 20 ml with H₂0 and injected on the column which then was eluted with a gradient of 40-60% (CH₃CN in water with 0.1% TFA) 10 ml/min during 50 min at 40 C. The peptide containing fractions were collected. The purified peptide was lyophilized after dilution of the eluate with water.

HPLC: (method A1): RT=45.5 min LCMS: m/z=792.9 (M+5H) 5+, 990.9 (M+4H) 4+, 1320.9 (M+3H) 3+Calculated (M+H)+=3959.9.

### Testing

Rats have been shown to like alcohol when offered access to choose between alcohol and water. Certain strains of rats are alcohol dependent and consume large amounts of alcohol if given the choice. It has been found that treatment with liraglutide significantly reduces the alcohol intake in rats compared with vehicle treated rats offered to choose between alcohol and water.

### Example 2

### Effect of Liraglutide on Alcohol Consumption in Normal SPD Male Rats

### Description of Assay:

Normal male Sprague Dawley rats were housed individually and fed normal rat chow. Each rat had access to two drinking bottles, one of them containing water and one of them containing a 10% alcoholic drink (diluted Toffee liquor from ALDI®). The rats were acclimatised to the alcoholic drink for a least one week. Consumption of water and the alcoholic drink were continuously recorded on line using the BIOdaq food and water intake monitoring system http://www.bodaq.com/®.

Basal water and alcoholic drink consumption was recorded on a 24 h daily basis. After this had stabilised, consumption of water and alcoholic drink was recorded over a 24 h period. The rats were divided in two groups, the first group assigned to receiving vehicle and the second group assigned to receiving liraglutide. The rats were dosed subcutaneously with either vehicle or a liraglutide solution (0.3 mg/ml administered by Novopen®). The Liraglutide dosed rats received 30 μg/kg liraglutide. Water and alcohol consumption were recorded for 24 h after injection. The 24 h accumulated consumption of water and alcohol solution was compared before and after administration of vehicle and liraglutide.

### Results:

The results are shown in Table 1. No difference was found in 24 h water and alcohol intake after treatment with vehicle. In contrast treatment with Liraglutide significantly and selectively reduced intake of alcoholic drink and not water.

TABLE 1
Accumulated fluid intake (g) over 24 h in mice administered vehicle
or liraglutide

| | n | Mean ± SEM |
|---|---|---|
| Water before vehicle | 8 | 11.1 ± 1.9 |
| Water after vehicle | 7 | 13.1 ± 2.4 |
| Alcohol before vehicle | 8 | 23.5 ± 1.8 |
| Alcohol after vehicle | 8 | 24.4 ± 2.8 |
| Water before Liraglutide | 7 | 11.2 ± 0.9 |
| Water after Liraglutide | 7 | 14.2 ± 1.7 |
| Alcohol before Liraglutide | 8 | 25.6 ± 0.6 |
| Alcohol after Liraglutide | 8 | 16.1 ± 1.3*** |

Paired t-test.
***represents p < 0.001

Numerous modifications and variations of the present invention are possible in light of the above teachings. It is therefore to be understood that within the scope of the appended claims, the invention may be practiced otherwise than as specifically described herein. In particular, while certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for treating alcoholism or drug addiction, said method comprising administering to a subject in need thereof a therapeutically effective amount of a GLP-1 agonist.

2. A method according to claim 1, said method comprising administering to a subject in need of treatment for alcoholism simultaneously or sequentially to administration of said GLP-1 agonist, another therapeutic agent selected from the group consisting of:
a. disulfiram, calcium carbimide, naltrexone, nalmefene, acamprosate, and benzodiazepines.

3. The method according to claim 2, wherein the GLP-1 agonist is a GLP-1 peptide comprising no more than ten amino acid residues which have been exchanged, added or deleted as compared to GLP-1 (7-37).

4. The method according to claim 2, wherein the GLP-1 agonist
a) is a DPPIV protected GLP-1 peptide;
b) is DPPIV stabilised;
c) comprises an Aib residue in position 8.

5. The method according to claim 2, wherein the GLP-1 agonist is selected from the group consisting of liraglutide, semaglutide, taspoglutide, albiglutide and dulaglitide.

6. The method according to claim 2, wherein the GLP-1 agonist has an in vivo plasma elimination half-life of at least 10 hours in man.

7. The method according to claim 2, wherein the GLP-1 agonist has chronic plasma exposure.

8. The method according to claim 13, wherein the drug addiction comprises addiction to a drug selected from the group consisting of: stimulants; sedatives and hypnotics; opioid analgesics; and opiates.

9-12. (canceled)

13. A method according to claim 1, said method comprising administering to a subject in need of treatment for drug addiction simultaneously or sequentially to administration of said GLP-1 agonist another therapeutic agent selected from one of the following groups:
i) amphetamine, methamphetamine, cocaine and caffeine;
ii) sedatives and hypnotics;
iii) opioid analgesics; and
iv) opiates.

14. A pharmaceutical composition comprising a GLP-1 agonist and another therapeutic agent selected from the group consisting of: disulfiram, calcium carbimide, naltrexone, nalmefene, acamprosate, and benzodiazepines.

15. A pharmaceutical composition comprising a GLP-1 agonist and another therapeutic agent selected from one of the following groups:
j) amphetamine, methamphetamine, cocaine and caffeine;
v) sedatives and hypnotics;
vi) opioid analgesics; and
vii) opiates.
